(19) United States
(12) Patent Application Publication
(10) Pub. No.: US 2026/0174203 A1
(43) Pub. Date: Jun. 25, 2026

(54) IDENTIFICATION MARK AND A METHOD FOR MANUFACTURING THE IDENTIFICATION MARK

(71) Applicant: Accenture Global Solutions Limited, Dublin (IE)

(72) Inventors: Lavinia Andreea DANIELESCU, Seattle, WA (US); Eric Michael GALLO, Moretown, VT (US); Aditi MAHESHWARI, Boston, MA (US); Wade INGRAM, San Francisco, CA (US)

(73) Assignee: Accenture Global Solutions Limited, Dublin (IE)

(21) Appl. No.: 18/999,764

(22) Filed: Dec. 23, 2024

(51) Int. Cl. A45C 3/00; A45C 3/06; B33Y 10/00; B33Y 80/00; C12N 1/14; D06P 1/00; D06P 3/32; D06P 5/20

(52) U.S. Cl. CPC A45C 3/001; A45C 3/06; B33Y 10/00; B33Y 80/00; C12N 1/14; D06P 3/32; D06P 5/2061; D06P 1/0096

(57) ABSTRACT

An identification mark and a method for manufacturing the identification mark is disclosed. The method includes providing a block of mycelium and integrating an NIR florescent dye into a surface of the block to define a first pattern in the surface of the block of mycelium. Once the NIR florescent dye is inserted, the NIR florescent dye is allowed to dry. Thereafter, the block of mycelium with dried NIR florescent dye is compressed into a thin sheet of mycelium leather and the compression results in distorting the shape of the first pattern into a second pattern. The created second pattern is the identification mark readable by the NIR camera and an illumination source.

### TECHNICAL FIELD

Various embodiments described herein relate generally to an identification mark and methods for manufacturing the identification mark.

### BACKGROUND

With ever-increasing technology demand, there are high expectations for product authentication. Authentication can be ensured by embedding metadata directly into the product. The authentication of luxury or specialized products can be achieved by embedding fluorescent dye-based identification marks into products. Fluorescent dyes are chemical compounds which, on exposing to a specific wavelength of light, emit light at a different wavelength. These fluorescent dye-based identification marks or tags not only function as an authentication marker for specialized products such as products made from mycelium or bio-based leathers but can also allow for embedding information into the product and support interactivity or customizability.

### SUMMARY

Implementations of the present disclosure are generally directed to embedding an identification marker in a product for authentication and interactivity. More particularly, implementations of the present disclosure are directed to methods for creating a sheet of mycelium leather bearing an identification mark visible by a near infrared (NIR) camera.

In general, innovative aspects of the subject matter described in this specification provide methods for creating a sheet of mycelium leather bearing an identification marking visible by a near infrared (NIR) camera. The methods include providing a block of mycelium and integrating an NIR florescent dye into a surface of the block to define a first pattern in the surface of the block of mycelium. Once the NIR florescent dye is inserted, the NIR florescent dye is allowed to dry. Thereafter, the block of mycelium with dried NIR florescent dye is compressed into a thin sheet of mycelium leather and the compression results in distorting the shape of the first pattern into a second pattern. The created second pattern is the identification mark readable by the NIR camera and an illumination source.

Furthermore, another method discloses creating a product from the sheet of mycelium leather bearing a marking visible by the near infrared (NIR) camera and an appropriate emission source to excite the dye. The method includes providing the block of the mycelium and integrating the NIR florescent dye into a surface of the block to define the first pattern in the surface of the block of mycelium leather. The NIR florescent dye solution is then allowed to dry. Once the NIR florescent dye solution dries up, the block of mycelium with dried NIR florescent dye is compressed into a thin sheet of mycelium leather. The compression distorts the shape of the first pattern into the second pattern. The thin sheet of mycelium leather is then cut into segments for assembly into a product. A segment of mycelium leather is then cut into a size and shape such that subsequent assembly of the product positions the second pattern at a predetermined location and a predetermined orientation on the product. The method includes assembling the segments in a manner that the second pattern appears in the predetermined location and/or predetermined orientation of the product.

It is appreciated that methods in accordance with the present disclosure can include any combination of the aspects and features described herein. That is, the methods in accordance with the present disclosure are not limited to the combinations of aspects and features specifically described herein, but also include any combination of the aspects and features provided.

The details of one or more implementations of the present disclosure are set forth in the accompanying drawings and the description below. Other features and advantages of the present disclosure will be apparent from the description and drawings, and from the claims.

### BRIEF DESCRIPTION OF DRAWINGS

Various embodiments in accordance with the present disclosure will be described with reference to the drawings, in which:

FIG. 1 illustrates a process of embedding an identification mark into a sheet, in accordance with implementations of the present disclosure;

FIG. 2 illustrates a process to integrate an identification mark into a sheet and to identify the embedded identification mark from the sheet, in accordance with implementations of the present disclosure;

FIG. 3A illustrates emission of photons from the embedded identification mark, in accordance with implementations of the present disclosure;

FIG. 3B illustrates a fluorescent spectrum, in accordance with implementations of the present disclosure;

FIG. 3C illustrates a process of optical identification of the embedded identification mark, in accordance with implementations of the present disclosure;

FIG. 4 illustrates a product into which the identification mark is injected, in accordance with implementations of the present disclosure;

FIG. 5A is a flow diagram that presents an example method for creating a sheet of mycelium leather bearing a marking visible by a NIR camera, in accordance with implementations of the present disclosure;

FIG. 5B is a flow diagram that presents an example method for creating the product from the sheet of mycelium leather bearing a marking visible by the NIR camera, in accordance with implementations of the present disclosure;

FIG. 5C is a flow diagram that presents an example method to produce a 3D printed dentification mark;

FIG. 6 is a layout of cutouts in leather to make a tote bag; and

FIG. 7 illustrates a process of creation of biological pattern, in accordance with an embodiment of the present disclosure;

Like reference numbers and designations in the various drawings indicate like elements.

### DETAILED DESCRIPTION

In the following description, various embodiments will be illustrated by way of example and not by way of limitation in the figures of the accompanying drawings. References to various embodiments in this disclosure are not necessarily to the same embodiment, and such references mean at least one. While specific implementations and other details are discussed, it is to be understood that this is done for illustrative purposes only. A person skilled in the relevant art will recognize that other components and configurations may be used without departing from the scope and spirit of the claimed subject matter. Reference to any “example” herein (e.g., “for example”, “an example of”, by way of example” or the like) are to be considered non-limiting examples regardless of whether expressly stated or not.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only and is not intended to further limit the scope and meaning of the disclosure or of any exemplified term. Likewise, the disclosure is not limited to various embodiments given in this specification.

Without intent to limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, technical and scientific terms used herein have the meaning as commonly understood by one of ordinary skill in the art to which this disclosure pertains. In the case of conflict, the present document, including definitions will control.

The term “comprising” when utilized means “including, but not necessarily limited to”; it specifically indicates open-ended inclusion or membership in the so-described combination, group, series and the like.

The term “a” means “one or more” unless the context clearly indicates a single element.

“First,” “second,” etc., re labels to distinguish components or blocks of otherwise similar names but does not imply any sequence or numerical limitation.

“And/or” for two possibilities means either or both of the stated possibilities (“A and/or B” covers A alone, B alone, or both A and B take together), and when present with three or more stated possibilities means any individual possibility alone, all possibilities taken together, or some combination of possibilities that is less than all of the possibilities. The language in the format “at least one of A . . . and N” where A through N are possibilities means “and/or” for the stated possibilities (e.g., at least one A, at least one N, at least one A and at least one N, etc.).

It should also be noted that in some alternative implementations, the functions/acts noted may occur out of the order noted in the figures. For example, two steps disclosed or shown in succession may in fact be executed substantially concurrently or may sometimes be executed in the reverse order, depending upon the functionality/acts involved.

Specific details are provided in the following description to provide a thorough understanding of embodiments. However, it will be understood by one of ordinary skill in the art that embodiments may be practiced without these specific details. For example, systems may be shown in block diagrams so as not to obscure the embodiments in unnecessary detail. In other instances, well-known processes, structures, and techniques may be shown without unnecessary detail in order to avoid obscuring example embodiments.

The specification and drawings are to be regarded in an illustrative rather than a restrictive sense. It will, however, be evident that various modifications and changes may be made thereunto without departing from the broader spirit and scope of the invention as set forth in the claims. To check the authenticity of a product or to access a product's details like its features, characteristics and price, generally printed tags or digital tags (tags may interchangeably be referred to as an identification mark) are attached to the product. However, the conventional identification marks are easy to reproduce and easy to tamper with and there have been incidents where even digital identification marks or QR codes in physical spaces are sometimes replaced or modified. Hence, counterfeit products can be a significant problem for brands, especially for high end goods, such as leather clothing and accessories. Original products are easily reproduced inauthentically and printed with fake brand logos that look nearly identical to the authentic brand logos of the original products. These products are then sold as authentic products to consumers, who have no way of verifying their authenticity before purchase. Further, tampering of QR codes, at times, may give away data to unwanted parties, which can lead to malicious links and viruses. Thus, the problem associated with the product is related to tampering of physical or digital identification marks on the products. Also, if identification marks are loosely attached, they can easily be removed, while if pasted on products, it negatively impacts the aesthetic appearance of the product or could damage the product.

In light of this, implementations of the present disclosure propose a system and method in which the tags are embedded in the surface of the product. Further, the present disclosure discloses the use of identification marks or tags (interchangeably referred to as identification marks) in an invisible format, i.e., not visible to the naked eye that do not impact the aesthetics of the product and are more difficult to reproduce as they are embedded at the time of manufacture within the product itself. Such identification marks fulfill the need to embed metadata directly into physical products. The metadata may include descriptive information about the product's identity and authenticity, product origin, function, price of the product, a link to a URL, etc. Hence, in one embodiment, the method disclosed herein addresses the issue of counterfeit leather products by embedding invisible identification markers within the authentic leather material. Such identification markers, when detected, provide a verifiable link to the original product's identity, ensuring the authenticity of the product.

To embed metadata into an invisible digital form into an alternative leather goods product, a user may fabricate a product (interchangeably referred to as an object), with the identification mark during fabrication of the mycelium block that is used to make the product. The hidden identification mark/tag may be in the form of one or more invisible marks, for example an invisible code. The identification mark/tag fabricated in the product may use fluorescent dye, that upon incidence of a particular wavelength light, enables the tag to emit photons at a specific near-infrared wavelength. Such identification marks are not visible to the naked eye and may be viewed using high contrast infrared cameras, reducing the possibility of tampering with or creating an inauthentic product. Thus, the identification marks are unobtrusive, i.e., as they do not appear on the product in the visible light spectrum seen by the naked eye, these identification marks do not change the product's shape, appearance, or function.

In view of this, implementations of the present disclosure propose a technique to develop invisible identification marks in mycelium leather, which are visible to special cameras and emission sources. In other words, the present disclosure discloses a method for creating a sheet of mycelium leather bearing a marking visible by a near infrared (NIR) camera. The method includes providing a block of mycelium leather and integrating an NIR florescent dye into a surface of the block to define a first pattern in the surface of the block of mycelium leather. The method further includes, allowing the NIR florescent dye to dry. Once the dye dries, the block of mycelium leather with dried NIR florescent dye is compressed into a thin sheet of mycelium leather. The compression distorts the shape of the first pattern into a second pattern. The second pattern is the identification mark readable by the NIR camera.

Furthermore, the present disclosure discloses another method for creating a product from a sheet of mycelium leather. Once the mycelium leather sheet is prepared, as discussed above, the segments of mycelium leather are cut into a size and shape and manufactured to form the product, positioning the marker at a predetermined location and/or at a predetermined orientation on the product. The mycelium leather sheet used to manufacture the product includes a preprinted pattern of NIR dye.

The preprinted pattern of NIR dye or tag is embedded with metadata to enhance the user's experience. For example, when the user buys a mycelium leather purse, the manufacturer links the embedded fluorescent tag or identification marker to the user's name in their database or system to register the user.

In an example use case, the tag can be used to embed an identification number of a product, for example an identification of a wallet made of mycelium leather. Scanning the tag allows a user or a seller to authenticate the product. Hence, the embedded invisible tag enables product authentication. Further, the invisible tag may be used to embed other metadata related to the product, the manufacturer or the user, and various such alternative use cases are described below in the present disclosure.

Mycelium leather, which is made from the root-like structure of mushrooms, is a sustainable alternative to traditional animal-based leather. To produce mycelium leather, fungal cells are combined with sawdust and organic matter. The mycelium develops into dense networks, creating soft, foam-like blocks. Once fully grown, the mycelium is harvested, shaped into mycelium blocks, and the leftover substrate is composted. The preparation process begins by drying and crushing agricultural waste, which is then mixed with a formulated composition of nutrients. These formulations contain necessary carbon, nitrogen and phosphate sources to support the growth of fungal spores. After adding adequate water and stirring to create a homogeneous mixture, the medium is packed into containers for sterilization. Once sterilized, fungal spores are introduced to the growth medium, and the fungus begins to grow. The mycelium, which is the root structure of the fungus, develops as part of the organism alongside other components such as the fruiting body and hyphae. Once the mycelium has sufficiently grown, it is harvested and processed into sheets of mycelium leather. For example, fungal species such as Penicillium chrysogenum, and Fomes fomentarius, may be used to produce the leather, though the method is not limited to these species. Thus, mycelium leather, which is also called mushroom leather is manufactured from mycelium which is the branching structure of threads that makes up fungus colonies, and out of which mushrooms grow. Mycelium grows by breaking down molecules like biopolymers in materials such as wood and plant waste by absorbing the smaller components. Mycelium, the primary structure of fungi, develops from fine threads that create a dense underground network. Over time, these threads fuse to each other, eventually forming a solid, foam-like material. Its branched cellular structure gives mycelium a natural strength and durability, which makes it an effective alternative to leather.

Mycelium may be 3D printed, where mycelium is mixed with pulped paper, coffee grounds, or other biowaste and then cold extruded into the desired shapes. NIR fluorescent dyes or proteins may be incorporated into the printing media that is extruded, creating fluorescent tags within the bulk product. The 3D end-product may then be compressed into mycelium leather or remain as the 3D structure that was printed, depending on the desired application.

Fluorescent dyes may be used to create fluorescent tags within the material that are chemically absorbed to the mycelium and remain in place to be functional. The mycelium uptake can be as high as hundreds of mg dye per gram of mycelium. As an example, two model species that may be absorbed without chemical modification into mycelium are Penicillium chrysogenum, and Fomes fomentarius.

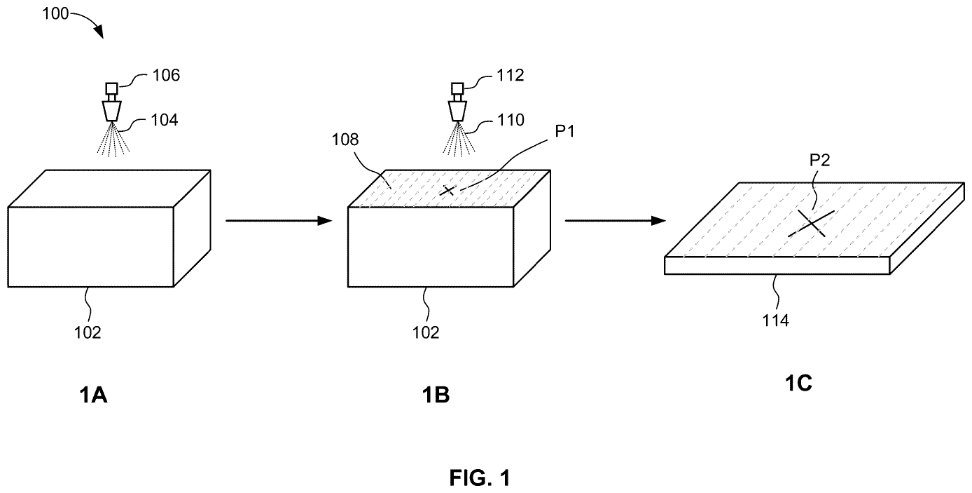

FIG. 1 illustrates a method 100 of embedding an identification mark into a sheet of mycelium, in accordance with implementations of the present disclosure.

Initially, at step 1A a block of mycelium 102 is selected for embedding an identification mark. The term “block of mycelium” refers to mycelium material in its raw or intermediate state, which will eventually be shaped into specific products. The block of mycelium 102 may be pre-treated. For example, at least one surface of the block of mycelium leather 102 is pre-treated with a chemical 104. In some examples, the chemical 104 is used for pre-treating the surface of the block of mycelium 102 to improve the rate of absorption of the NIR florescent dye to the block of mycelium 102. The chemical 104 may include but is not limited to, 0.1M H₂SO₄, NaOH, crosslinked PEI (polyethylenimine), some acids, some bases may be allowed to treat the surface for greater dye absorption and adhesion. In one implementation, the chemical 104 may be applied on the surface of block of mycelium 102 using a nozzle 106, wherein the nozzle 106 may be operated using a robotic arm, for example. Pretreating the surface by inkjet printing in precise locations may tune the surface to readily absorb the dye molecules in those pretreated regions. The pretreatment process may enhance the contrast of the NIR dyes.

Upon pretreating the surface of the block of mycelium 102 with the chemical 104, a pretreated surface 108 is formed on the mycelium 102. Then, in one embodiment of the present disclosure, an NIR fluorescent dye (NIR fluorescent dye may interchangeably referred to as dye) 110 is integrated to the pretreated surface 108 of block of mycelium 102 to define a first pattern P1. The NIR fluorescent dye 110 is prepared by mixing the fluorescent dye into a solvent. The integration may refer to applying the fluorescent dye along with solvent on the pretreated surface 108 by one of the exemplary techniques, which may be but not limited to 3D printing, spraying, injecting the NIR fluorescent dye 110 to the pretreated surface of the block of mycelium 102. In one embodiment, a nozzle 112 controlled using a robotic arm is used for applying the fluorescent dye 110 on the pretreated surface 108 of block of mycelium 102, as shown at step 1B. In some examples, the injection of the NIR fluorescent dye 110 depends upon the density of the mycelium leather. Integrating the NIR fluorescent dye 110 to a surface 108 of the block of mycelium 102 defines the first pattern P1 on the surface 108 of the block of mycelium 102. The density of the block 102 and the properties of the dye 110 results in a unique combination. The solution viscosity, which is determined both by the solvent viscosity and the dye to solvent ratio determines the absorption depth, along with the surface interaction between the mycelium medium and the dye solution. Higher viscosity solutions will have a lower penetration depth, while lower viscosity solutions will have a deeper penetration depth. Stronger surface interaction (for instance using a water-based solution on a hydrophilic surface) will also decrease depth, this is controlled by the pretreatment of the material.

Upon integrating the dye 110 to the pretreated surface 108 of the block of mycelium 102, the NIR florescent dye solution 110 may be left for drying. Once the dye 110 dries, the block of mycelium 102 is compressed with the dried NIR florescent dye 110 to form a thin sheet 114 of mycelium 102, as shown at step 1C. Compressing the dye-created pattern embedded in the sheet ensures that the fluorescent inks are not erodible and that the pattern is included within the mycelium 102 from the beginning of the production process. Compression within the tag ensures that the pattern cannot be reproduced by a counterfeiting agency by simply adding the pattern on top of the mycelium leather tag, for example. Further, compressing the mycelium 102 with the first pattern P1 forms a second pattern P2. The second pattern P2 is an identification mark (may be interchangeably known as an NIR tag or NIR fluorescent tag), readable by an NIR camera, produced on the thin sheet 114 of the mycelium 102. The identification mark P2 may be a brand logo or symbol or 2D matrix code, barcode, serial number and/or the like. Therefore, the sheet 114 of mycelium 102 integrated with the NIR florescent dye 110 as a second pattern P2 of the identification mark is generated. Thus, between providing the block and integrating the dye, the surface of the block is pretreated with the chemical 104 that controls the rate of absorption of the NIR fluorescent dye 110 into the block of mycelium 102. The dye is integrated by inkjet printing of the NIR fluorescent dye 110 onto the surface of the block of mycelium 102.

In another embodiment, mycelium may be 3D printed, where mycelium is mixed with pulped paper, coffee grounds, or other biowaste and then cold extruded into the desired shapes. Then the NIR fluorescent dyes or proteins may be incorporated into the printing media that is extruded, creating fluorescent tags within the bulk of the product to produce the first pattern P1. The 3D end-product is then compressed into mycelium leather or remains as the 3D structure that was printed, depending on the desired application. The compression forms the second pattern P2.

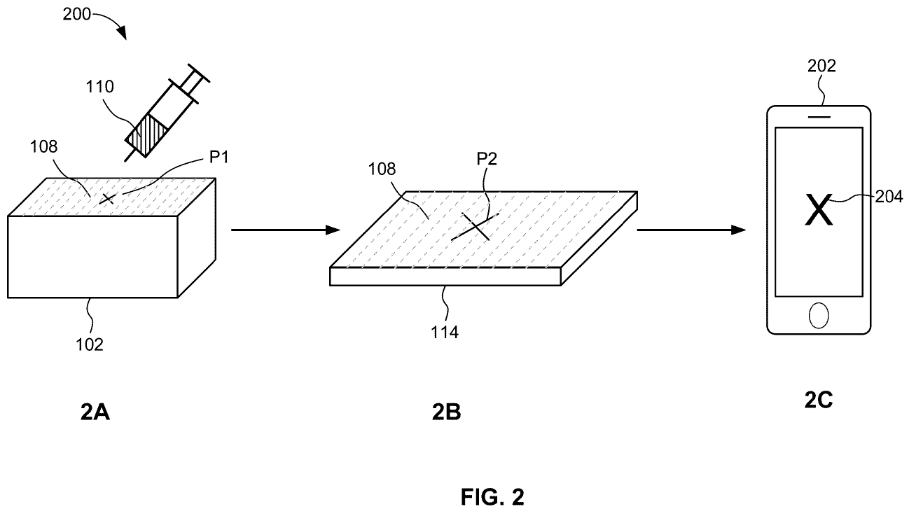

FIG. 2 illustrates a process 200 of integrating an identification mark into the sheet and identifying the embedded identification mark from the sheet, in accordance with implementations of the present disclosure.

To integrate an NIR fluorescent dye in the form of an identification mark P1, in the sheet of mycelium leather, the NIR fluorescent dye 110 is injected into an area of the block below the surface 108 of the block of mycelium 102, as shown at step 2A. In some examples, the NIR florescent dye 110 is injected using robotics at precise locations on the surface 108 of the blocks of mycelium 102. Upon injecting the NIR fluorescent dye 110, the first pattern P1 is formed on the surface 108 of the block of mycelium 102. After the formation of the first pattern P1, the NIR fluorescent dye 110 is allowed to dry. Further, the block of mycelium 102 is compressed with dried NIR fluorescent dye 110 into a thin sheet 114 of mycelium 102, as shown at step 2B. The compression of the first pattern P1 distorts the shape of the first pattern P1 into the second pattern P2. In some examples, the second pattern P2 may be the identification mark P2 readable by the NIR camera. The identification mark P2 is “X”, where X may be a brand logo or symbol or matrix code or barcode, serial number and/or the like. As an example, it should be noted that compression occurs along the z-direction and is generally moderate. As a result, the block expands primarily in the x-y plane, with minimal expansion in the z-direction. As a result, patterns may not distort much and there is possibility to have the second pattern P2 be similar to the first pattern P1. In the case that the first pattern P1 distorts, the first pattern P1 changes into the second pattern P2. Accordingly, these patterns can be pre-designed based on the mycelium block thickness and density to account for distortions during compression, as distortion will be repeatable within a margin of error. In another example, the integrating of the dye 110 includes inkjet printing of the NIR fluorescent dye 110 onto the surface 108 of the block of mycelium 102. Inkjet printing generally has high throughput, which primarily leaves the dye on the surface 108, from where the dye is chemically absorbed into the block of mycelium 102. Inkjet printing deposits small amounts of dye with high resolution, to create matrix codes, barcodes, serial numbers, etc. In another example, robots precisely inject fluorescent dyes 110 at the precise locations in the block of mycelium 102 before the block may be compressed into a sheet. In some examples, the compression process may add constraints to areal resolution of the applied patterns due to the distortion. It can pattern logos for simple branding authentication, as well as barcodes, serial numbers, or other authentication tags. FIG. 2 is described in conjunction with FIG. 1. Upon the formation of identification mark P2 (the second pattern), on the surface 108 of block of the mycelium 102 or in a mycelium product, a user may use a user device 202, such as a cellphone with an added NIR camera, to scan the identification mark 204. As described, the compression of the block of mycelium 102 with the fluorescent dyes 110 (forming the first pattern P1) forms the second pattern P2. Hence the second pattern P2 emerges organically and whatever pattern emerges organically is used as a unique identifier of the product. In one embodiment of the present disclosure, the second pattern P2, which is organically created through compression, may be scanned and registered in a database by associating it with the metadata of the product stored in the database, wherein the metadata may include, but is not limited to, a unique identifier of the product or the manufacturer, price of the product, owner's name of the product, etc. In one embodiment, to store the second pattern P2 with the metadata, the second pattern P2 may be identified and registered using a light source and a detector. That is, light from a light source is made to incident on the second pattern P2 formed on the product and light reflected from the fluorescent dyes 110 is read to register the second pattern P2. Then the second pattern P2 is tagged with the metadata and stored in a cloud for further use by an end user. It is to be noted that the second pattern may be scanned and registered in the database using a dedicated application. Further, an end user may scan the second pattern produced on the product using a dedicated application, a client application for example, to verify the authenticity of the product.

In one embodiment of the present disclosure, the end user may use the user device 202 for scanning the second pattern P2, which may be referred to as a NIR fluorescent tag P2, and authenticate the product, for example, as shown at step 2C. The user device 202 may be for example but not limited to an NIR scanner, a camera with a processor, and a smartphone with NIR scanning capability. In some examples, the NIR fluorescent tag P2 (interchangeably referred to as identification mark) in mycelium leather may be scanned using the user device 202 (may interchangeably be referred to as NIR camera, NIR imaging apparatus) using NIR technology, where the user device 202 may be of one or more mobile camera with specific additional filters, NIR camera, NIR scanner, NIR detector or the like. NIR is a region of the electromagnetic spectrum that has unique properties for characterizing materials. In the electromagnetic spectrum, the NIR region is between 700 to 2500 nanometers (nm).

In an example, the NIR technology may be used to scan the NIR tag (interchangeably referred to as authentication tag/identification mark) P2. The NIR tag P2 is embedded in the mycelium leather, such that the mycelium leather product appears opaque and unmodified under visible light but reveals the NIR tag P2 under near-infrared light.

In an example, the NIR tags P2 may be scanned by the user device 202, which may be an NIR imaging apparatus. The NIR imaging apparatus 202 may be for example an NIR camera that operates by capturing NIR light emitted by the dye in the mycelium from the NIR tag P2 through a lens system specifically designed to optimize the transmission of wavelengths within the near-infrared range, approximately from 700 nm to 2500 nm. When light is incident on the NIR tag P2, the captured NIR radiation may be directed to a sensor array that is sensitive to the wavelength, converting the incident light into corresponding electronic signals. Further, the electronic signals are digitized and processed by an onboard imaging processor, utilizing techniques tailored for enhancing contrast and resolving fine details. A resulting digital image 204 (which is the second pattern P2) may be constructed by sampling the signals. Hence, the NIR camera 202 scans the NIR tag P2.

The NIR tag P2 is embedded or built into a mycelium good, i.e., mycelium leather, during the manufacturing of the mycelium leather.

In an example, modern cameras utilize focal plane arrays (FPAs) as their image sensors. These FPAs consist of an array of light-sensing pixels positioned at the focal plane of the camera lens. FPAs are commonly used for imaging purposes, such as capturing photos or video imagery. When photons strike the individual pixels (detectors) within the FPA, they generate an electrical charge, voltage, or resistance. This process occurs via the photoelectric effect. The generated electrical signal is typically stored in a capacitor associated with each pixel. The accumulated charge represents the amount of incident radiation. FPAs are based on silicon, which are sensitive to both the visible and near-infrared (NIR) spectra. As a result, the typical sensitivity curves for each pixel resemble those of silicon photodiodes. The sensitivity curve for a silicon photodiode provides valuable information about the pixel's response to different wavelengths. The actual detection of NIR radiation by the digital cameras depends on the spectral transmittance of color filters and optics between the lens and the detector.

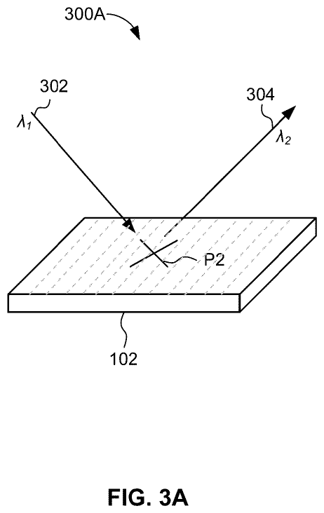

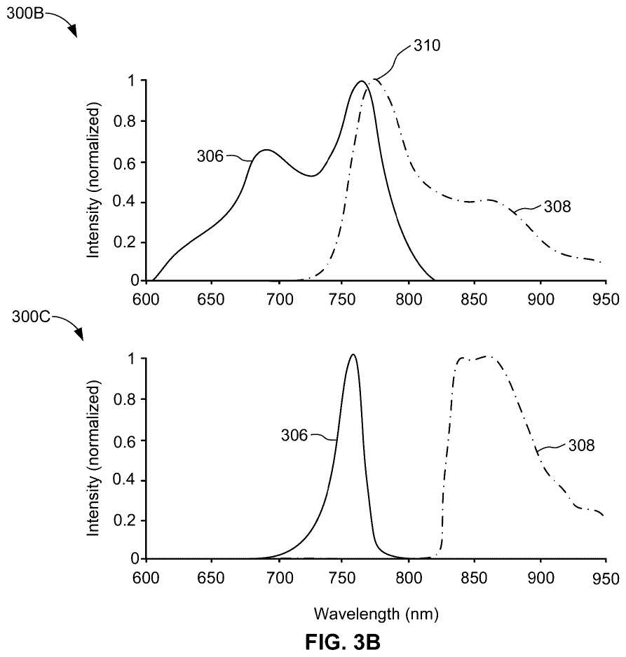

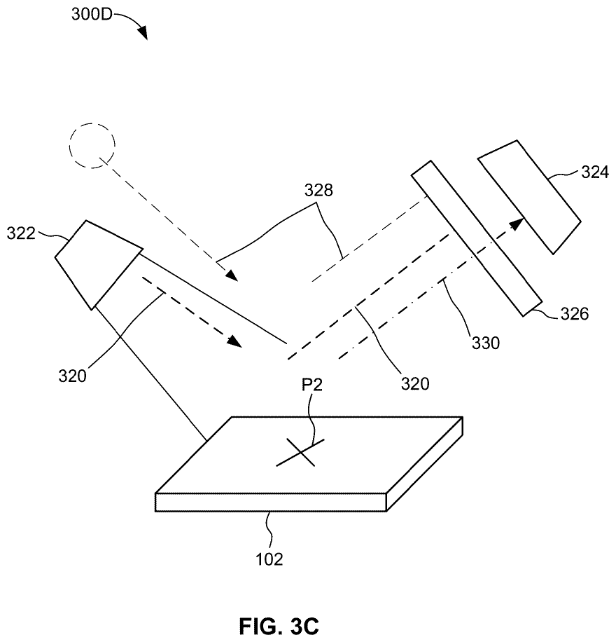

FIG. 3A illustrates emission of photons from the embedded identification mark, in accordance with implementations of the present disclosure.

As described, injection of the NIR fluorescent dye 110 into the mycelium block 102 forms the second pattern P2 (identification mark or the NIR tag) on the surface 108 of the block of mycelium 102. The embedding of the second pattern P2, which is formed due to the NIR fluorescent dye 108, shifts the wavelength of IR radiation when incident on the second pattern P2. The depth and density of the dye after application and drying determines whether the fluorescence signal is weak or robust for a tag.

To read the embedded second pattern P2, the NIR fluorescent material is excited using light from a light source. In order to achieve this, firstly, a light source is selected. A light source may include but is not limited to a high-power LED or broad band source with a narrow band filter at the emission wavelength on the image sensor. Secondly, the incident light on the NIR fluorescent tag P2 should have high power, whose peak wavelength should be as close to the material's peak excitation wavelength (for example, 763 nm). The tags may be visible to a camera with NIR sensitivity under many circumstances. The condition to be avoided is that there should not be much additional ambient NIR signal that drowns out the image and the excitation should be positioned such that the image sensor is not receiving full reflection but only a scattering of the excitation light. A higher power LED may be used that peaks at 760 nm and delivers power that excites the dye to emit at levels above ambient light. Thus, an incident high power light 302 of wavelength λ₁ is incident on the NIR fluorescent tags P2. This emits the light 304 of wavelength λ2, that is, the excitation light emitted from the NIR tag by the LED towards the camera. The camera uses filters, due to which the only wavelength range that can enter the camera corresponds to the emitted NIR wavelength light.

FIG. 3B illustrates a fluorescent spectrum, in accordance with implementations of the present disclosure. In an example, the first graph 300B illustrates both the excitation (absorption, shown by the continuous line 306) and emission (fluorescence, shown by the dotted line 308) spectra of the material. Unfiltered spectra reveals that the emitted fluorescence has a longer wavelength than the absorbed light. Specifically, when the material (of the fluorescent tag) is most excited at a wavelength of 763 nm, the peak 310 of the emitted light occurs at 775 nm. The 12 nm difference, known as the Stokes shift, allows to separate the excitation and emission signals for infrared (IR) image capture in the second graph 300C. However, due to spectral overlap, optical filtering methods are necessary to isolate these signals. Through a wavelength-specific filter, it is possible to enhance the recognition of fluorescent markers by minimizing interference from other wavelengths as shown in the second graph 300C. Because of the inherent properties of fluorescence, there exists an overlap between the higher-wavelength end of the excitation spectrum and the lower-wavelength end of the emission spectrum. This overlap, depicted in the first graph 300B, needs to be minimized to prevent the stronger excitation light from overpowering the weaker emitted fluorescence light. Failure to address this overlap would significantly diminish marker contrast.

To separate these signals, a long pass or bandpass filter may be used with a threshold wavelength or specific wavelength, which blocks any shorter or outside the band wavelengths from entering the camera.

FIG. 3C illustrates a process 300D of optical identification of the embedded identification mark, P2, in accordance with implementations of the present disclosure.

During the identification process, that is to read the embedded identification mark P2, light (excitation light) 320 from an incident light source 322 on the identification mark P2 embedded into the surface of the block of mycelium 102 is captured using a camera 324 of the user device 202, for example. In one embodiment, a long pass filter with a particular threshold wavelength (for example 830 nm) is used to block any wavelength below the threshold wavelength from entering the camera 324. Referring to FIG. 3C, the long pass filter 326 blocks excitation light 320 and ambient light 328, and only allows fluorescence light 330 from the NIR fluorescence dyes of the identification mark P2. Hence, only wavelength range that corresponds to the fluorescence from the identification marker P2 can enter the camera 324. Hence the long pass filter 326 filters out emission below 810 nm, allows the fluorescent light of approximately 820 nm to enter the camera 324. The captured fluorescent light 330 is used to reconstruct the image, that is the identification mark P2. It is to be noted that the FIG. 3C illustrates a dedicated light source 320 for producing the excitation light 320. However, a light source of the user device 202 may be used for exciting the identification pattern P2.

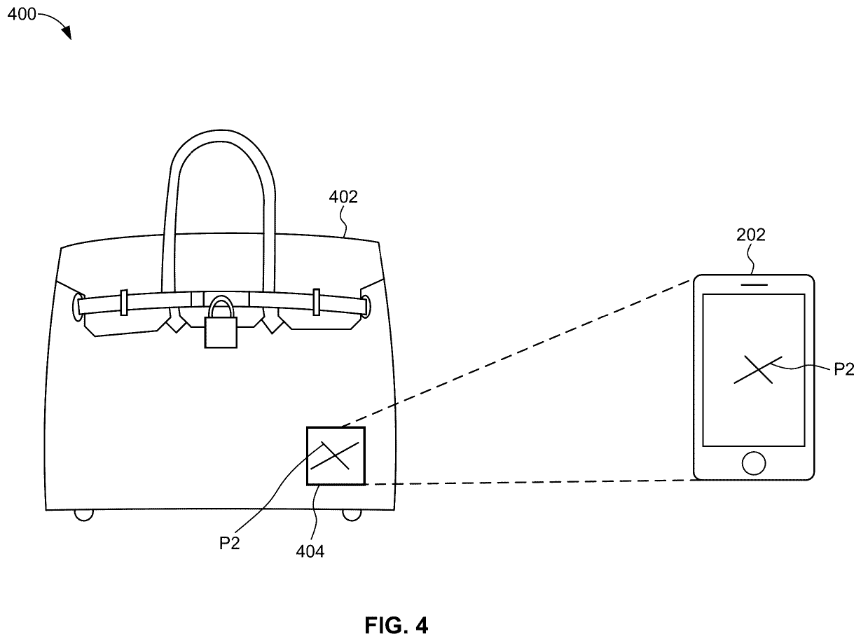

FIG. 4 illustrates a product with an identification mark, in accordance with implementations of the present disclosure.

FIG. 4 illustrates a product 402 with the identification mark P2, in accordance with an embodiment of the present disclosure. It is to be noted that the identification mark P2 may be embedded into the product 402 in multiple ways. In one example, the sheet of mycelium 102 along with the identification mark P2 is formed and then the sheet of mycelium 102 is cut into a predetermined shape to form the product 402 with the identification mark P2 at a predetermined location and orientation. In another example, a sheet 404 of predetermined size and shape is formed along with the identification mark P2 as described in the present disclosure and then the sheet 404 is embedded or stitched to the product 402 as shown. An end user may use his/her user device 202 to scan and authenticate the product 402 as shown in FIG. 4.

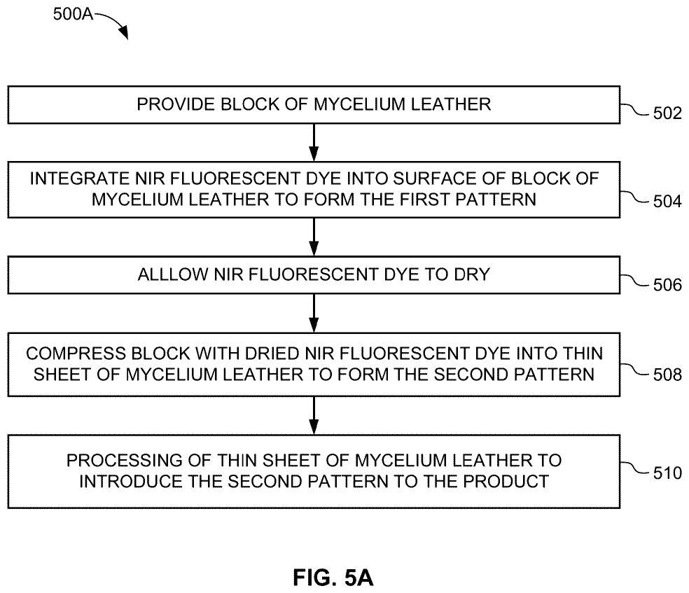

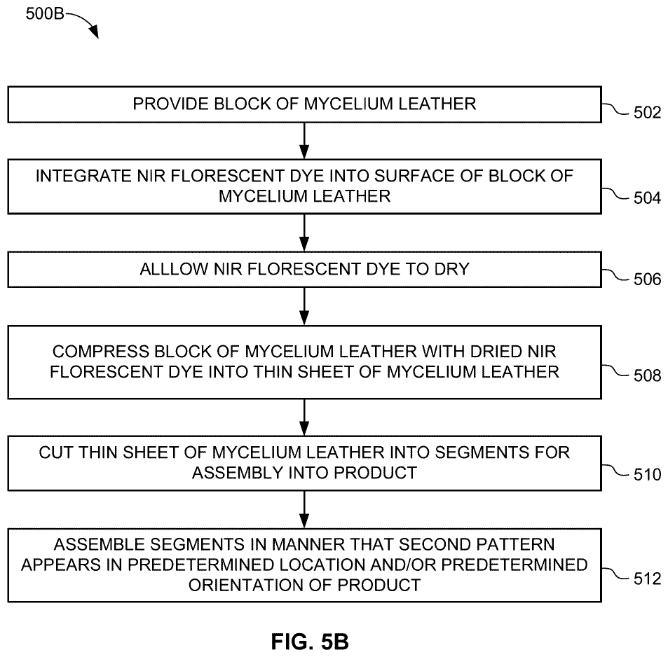

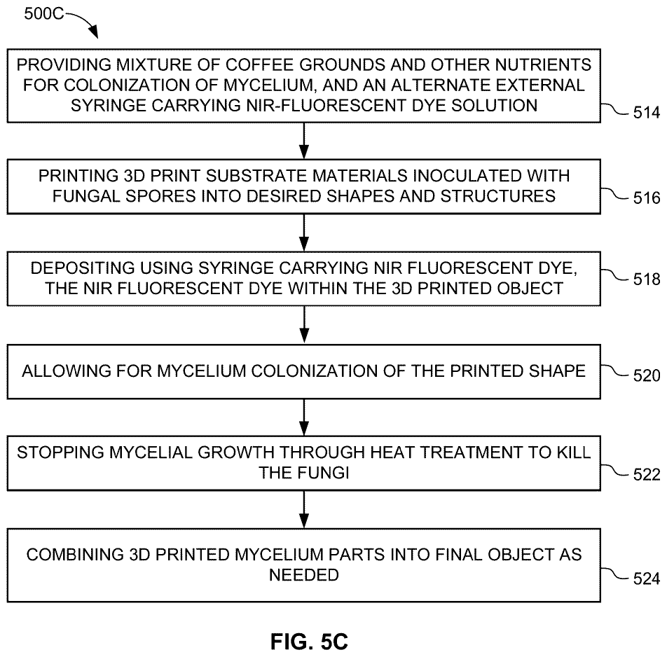

FIG. 5A is a flow diagram illustrating an example method 500A for creating the sheet of mycelium leather 114 bearing an identification mark visible by the NIR camera, in accordance with implementations of the present disclosure.

At step 502, the method 500A discloses providing a block of mycelium 102. The production techniques to create the block of the mycelium 102, is disclosed above with reference to FIG. 1 and is not repeated for the sake of brevity. The block of mycelium 102 may be manufactured using different techniques as recited in the present disclosure. In a non-limiting example, the size and shape of the mycelium block 102 suitable for processing into sheets of the mycelium leather 114 may be considered as having a length of 12-24 inches (approx.), a width of 12-24 inches (approx.), and a height of 4-8 inches (approx.). The dimensions of the block are based on the product 402 requirements. The invention is not limited to any particular size or shape of the block.

At step 504, the method 500A discloses integrating an NIR florescent dye 110 into a surface 108 of the block of mycelium 102 to form the first pattern P1. In an example, the NIR fluorescent dye 110 is integrated into an exposed surface of the block of the mycelium 102 to define a first pattern P1. Various figures here show integration into the top surface, but the present disclosure is not limited only to the top surface, however, and may be integrated into any surface. In an example, integrating the dye includes inkjet printing the NIR fluorescent dye 110 onto the surface of the block of mycelium 102. For better absorption of the NIR fluorescent dye 110, the surface of the block may be pretreated with a chemical that improves a rate of absorption of the NIR fluorescent dye 110 to the block of mycelium 102. The chemical used for pretreatment may be one or more of Penicillium chrysogenum, Fomes fomentarius and the like. Some non-exhaustive examples of the chemicals may include 0.1M H2SO4 , NaOH, crosslinked PEI (polyethylenimine), and other acids and bases.

In another example, the NIR fluorescent dye 110 is a combination of a dye and solvent that preferably have several properties. the NIR fluorescent dye 108 may fluoresce under appropriate illumination and appear visible on the NIR camera. Further, the NIR fluorescent dye 110 is absorbable into the mycelium 102. Furthermore, the fluorescent dye 110 may adhere to the injection point and hold its shape during subsequent leather processing. Non-limiting examples of the NIR fluorescent dye 110 include indocyanine green, DIC proprietary NIR fluorescent dye, terrylenimides, lumiprobe NIR fluorescent dye, although the present disclosure is not limited to any particular dye.

In some examples, the ratio of NIR fluorescent dye 110 to solvent may be optimized for better absorption. A non-limiting example of a dye/solvent ratio may vary from 0.05 mg/mL to 1 mg/mL of dye in solution, but the present disclosure is not limited to any particular ratio other than as may be needed to satisfy the presence as above. The block of mycelium referred to in the present disclosure is an example of a structural biomaterial that composes polysaccharides, for example but not limited to chitin. In an example, the NIR fluorescent dyes may adhere to chitin based on chemical affinity. This may be from ionic affinity and molecular reactions between the chitin and the dye, chitin has acetyl groups that ionically bond with anionic groups in dyes, for example: chitin AB161. Another example may be considered as metal ions that coordinate binding and chemical affinity. In another example, fungal staining chemistry known as PAS (Periodic acid-Schiff) affixes dyes to polysaccharides that have been broken down by periodic acid. The Schiff reagent may be replaced with NIR-fluorescing dyes instead to pattern the surface.

At step 506, the method 500A includes allowing the NIR florescent dye 110 to dry. In an example, upon the integration of the NIR florescent dye 110 to the surface of the block of mycelium 102, the NIR florescent dye 110 may be allowed to dry. In one implementation, the NIR florescent dye 110 may be dried using drying agents.

At step 508, the method 500A includes compressing the block of mycelium 102 with dried NIR florescent dye into a thin sheet of mycelium leather 114, wherein the compression forms the second pattern P2. In an example, the block of mycelium 102 with dried NIR florescent dye is compressed into a thin sheet of mycelium leather 114 using compression techniques such as a hydraulic press. However, the present disclosure is not so limited, and any other compression technique may be used. The compression transforms the block of mycelium 102 into a thin sheet of mycelium leather 114. The compression of the block of mycelium 102 into a sheet 114 reduces the thickness of the mycelium leather, for example to less than 0.1 inches. However, the present disclosure is not limited to any specific thickness.

At step 510, the method 500A discloses to process the thin sheet of mycelium leather to introduce a second pattern P2 to the product. As described, upon forming the thin sheet of mycelium leather 114 with the second pattern P2, the sheet is cut and molded to form the product with the second pattern P2 which may be used for authenticating the product, for example.

The compression technique may alter the length and width of the mycelium leather, such that the compression may cause the first pattern P1 to change into the second pattern P2. Since the second pattern P2 will be the one required by the manufacturer to be visible to an NIR camera, the first pattern P1 as established at step 504 may be designed with a specific shape that achieves the second pattern P2 upon compression. However, to the extent that compression does not cause a drastic shape change, the second pattern P2 may be identical to the first pattern P1.

FIG. 5B is a flow diagram that presents an example method 500B for creating the product 402 from the sheet of mycelium leather 110 bearing a marking visible by the near infrared (NIR) camera, in accordance with implementations of the present disclosure.

The initial steps 502 to 508 are similar to the method disclosed above in 500A and therefore not repeated for the sake of brevity. In essence, the method flow 500A discloses a method of integrating the fluorescent dye with the block of mycelium, which after drying is compressed to form a sheet of the mycelium leather with NIR fluorescent dye.

Upon the creation of the sheet of the mycelium leather 114, the method 500B discloses the creation of the mycelium product 402 from the sheet of the mycelium leather 114 using the following steps.

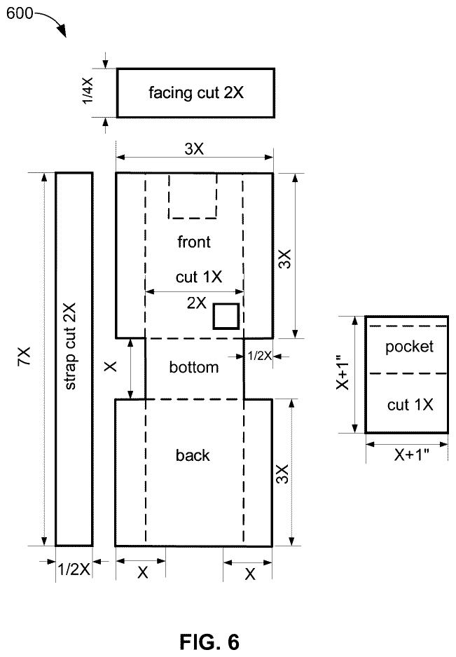

As is known in the art, sheets of leather or fabric are cut into segments of specific shape and sizes that are later connected into a final product. By way of non-limiting example, FIG. 6 shows a schematic of cuts in leather that are later assembled into a tote bag. At step 510, the method 500 includes cutting a thin sheet of mycelium leather 110 into appropriate segments for assembling into a product 402. These cuts may be made directly from the original compressed sheet from step 508, or from smaller swatches of the original compressed sheet in an intermediate cutting step.

In typical production methodologies, the orientation of the shapes as cut into the leather is not relevant, with the layouts often being optimized to limit the production of scrap. In step 510, the cutting segments of mycelium leather may include cutting into a size and shape such that subsequent assembly of the product 402 positions the second pattern P2 at a predetermined location and/or predetermined orientation on the product 402. Thus, with reference to product 402 in FIG. 4, the front panel of the bag is cut from the sheet of leather such that the second pattern P2 appears in the lower right-hand corner of the completed product 402. An accommodation in subsequent manufacturing may be to monitor the location of the second pattern P2 to ensure that the second pattern P2 appears at an appropriate location in the final product 402.

At step 512, the method 500B includes assembling the segments in a manner that the second pattern P2 appears at the predetermined location and/or predetermined orientation of the product 402.

By way of non-limiting example, if the product 402 is a mycelium leather purse, the mycelium leather may be cut and assembled, so the second pattern P2 appears at a predetermined location, such as adjacent to the normal logo on the purse, rather than in some haphazard location. Segments of leather bearing the second pattern P2 may be cut and sized to place the second pattern P2 at the desired location and/or orientation in the assembled product 402. In another non-limiting example, the orientation could be predetermined, e.g., oriented in parallel with the top of the product 402.

When the product 402 is viewed under appropriate illumination by an NIR camera, the second pattern P2 appears in the viewscreen of NIR camera. There are a variety of integration options at step 504. In some examples, a methodology may be to inject the NIR fluorescent dye 110 into the block of mycelium via manual or robot injection, as disclosed at step 504 in the method 500A. The injection may place the dye 110 at a depth below the surface that is deep enough, so the dye 110 may not visible (e.g., as a discoloration in the leather) under ordinary light but not so deep that the fluorescence may not sufficiently detectable by the NIR camera. A depth of about 0.3 mm to 1.2 mm may be appropriate, but the present disclosure is not so limited to any particular depth.

In an example, before printing the NIR fluorescent dye 110 onto the surface of the block, the dye and/or solvent may be selected to ensure a collective viscosity suitable for use with a known printing methodology. For example, the inkjet methodology may include a suitable viscous solvent for printing the identification mark on the pretreated mycelium leather surface of the block 102, with a chemical 104 that forms a proper NIR fluorescent tag, for example but not limited to a logo, matrix code, brand name etc. with the NIR fluorescent dye dissolved in the solvent. Non-limiting examples of such a chemical include crosslinked polyethylenimine, acrylic polymers, chitosan, polyvinylalcohol. acrylic polymers to be used as viscosity modifiers, which are natural biopolymers, but the present disclosure is not limited to any particular chemical.

A database of second patterns P2 may be maintained for specific products and/or vendors and compared against the image detected by the NIR camera. If the image matches the second pattern P2, in the desired location and/or orientation, the product is authentic. If no fluorescent image is present or if the image is incorrect or in the wrong location or orientation, the product 402 may be categorized as not authentic. Therefore, by incorporating NIR fluorescent dyes 110 into mycelium goods during the manufacture of mycelium goods, an authentication tag or identification mark P2 is created within the product 402 that is non-fungible. Moreover, the identification mark P2 does not visibly appear on the product 402 under normal lighting, and thus does not compromise the aesthetics of the product 402.

FIG. 5C is a flow diagram that presents an example method 500C to produce a 3D printed identification mark.

At step 514, the method 500C discloses a mixture of coffee grounds and other nutrients provided for colonization of mycelium. Alternatively, a syringe may be provided that carries NIR fluorescent dye solution.

At step 516, the method 500C discloses an inoculation process where a 3D printing material is first printed to form a substrate. During this printing process, the material is inoculated with fungal spores, which are integrated into the material to form the desired shapes and structures. The method enables the creation of substrates that can support fungal growth, allowing for the development of specific forms or patterns through biological processes. Thereafter, the incubation takes place in which the mycelium begins to spread throughout the substrate. The process is driven by optimal temperature and humid conditions dependent on the strain of mycelium being used.

At step 518, the method 500C discloses that the syringe carries the NIR fluorescent dye and deposits the NIR fluorescent dye within the 3D printed object. At step 520, the method 500C discloses allowing the mycelium colonization of the printed shape. Further, at step 522, the method 500C discloses that mycelium growth is stopped once the printed shape is achieved by treating the mycelium with heat treatment to kill the fungi.

At step 520, the method 500C discloses combining the 3D printed mycelium parts into a final object as per the requirements. Thus, obtaining a mycelium leather product with a 3D printed mark on it. In one embodiment, two printheads, one with mycelium culturing materials like coffee grounds, and the other printhead with NIR fluorescent dye may be used to print the structure onto which the mycelium will grow and into which the NIR dye will be incorporated in exact locations. In another embodiment, both the printheads may include coffee grounds and other nutrients for growing mycelium. However, only one of the printheads includes NIR-fluorescent dye mixed in with the coffee grounds and other materials to be able to print NIR-fluorescent media where desired in the 3D structure.

In one of the examples, the integration methodology may be stamping the NIR fluorescent dye 110 onto the surface of the block. In another example, the integration methodology may be stamping the NIR fluorescent dye 110 onto the surface of the block of mycelium leather.

In view of this, implementations of the present disclosure propose, instead of visible identification marks, tags (interchangeably referred to as an identification mark), the tags being in an invisible format, i.e.—not visible to the naked eye, which again gives an added advantage of discouraging attempts to tamper with or create inauthentic products as the tags may be scanned through specific wavelength scanners only. These tags fulfill the need to embed metadata directly into physical products. The metadata discloses descriptive information about the product's identity, origin, function, etc. To embed metadata into an invisible digital form, a user may fabricate a product (interchangeably referred to as an object), with the hidden identification mark during fabrication. The hidden identification mark may be in the form of one or more invisible marks, for example an invisible matrix code. The identification mark fabricated in the products may use fluorescent materials, such as fluorescent dyes, that enable each tag to emit light at a specific near-infrared wavelength. Such identification marks may be viewed using high contrast infrared cameras and are not visible by the naked eye.

In another embodiment, a multilayer stack of mycelium leather is compressed, with the mid layer having a fully modified variant that expresses NIR-fluorescent proteins during cultivation. The mid layer may be customized with small regions cut out to create a custom pattern (e.g., brand logos or symbols or barcodes or serial numbers and/or the like). The outermost layer of mycelium leather may be removed to a major extent to expose the fluorescent layer.

In an exemplary method of manufacturing mycelium leather, the sheet of mycelium leather sandwiches a layer of genetically modified NIR fluorescent mycelium between two mycelium leather sheets. The identification mark is cut out from the layer of the genetically modified NIR fluorescent mycelium to create a custom pattern.

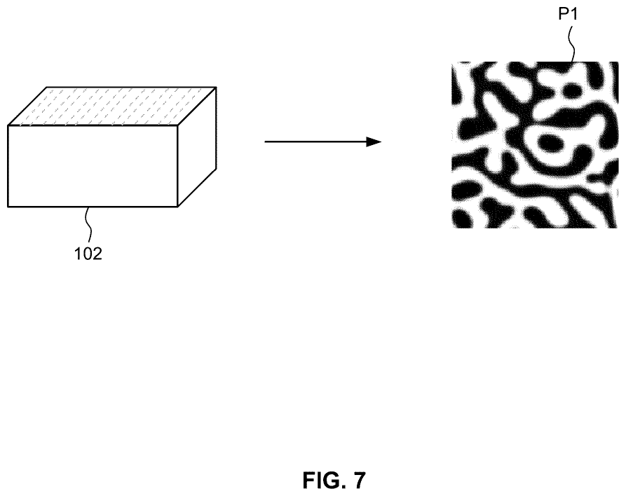

In another embodiment, a mixture of mycelium (e.g., A. niger, F. fomentarius, P. chrysogenum) and a genetically modified species that expresses an NIR-fluorescent protein can be combined to create a biological pattern, that may be used as a unique identifier for the product 402. A block of mycelium nutrients (sawdust, etc.) may be inoculated with an unmodified mycelium species as well as genetically modified mycelium that expresses NIR-fluorescent proteins. FIG. 7 illustrates a process for the creation of a biological pattern, in accordance with an embodiment of the present disclosure. When the block of mycelium 102 is inoculated with unmodified mycelium species and genetically modified mycelium that expresses NIR-fluorescent proteins, a fluorescent pattern may form from the growth of nonfluorescent mycelium (black pattern) and genetically modified NIR-fluorescent mycelium (white pattern) which creates the fluorescent pattern, the first pattern P1. Then upon compression of the mycelium block 102, the second pattern P2 is formed and the second pattern P2 is used to authenticate the product as described in the present disclosure.

As described, the identification mark in an invisible format, and methods of manufacturing the identification mark are disclosed. Such identification marks cannot be reproduced as they are embedded within the product itself during the manufacturing of the product. Such identification marks fulfill the need to embed metadata which may include descriptive information about the product's identity and authenticity, product origin, function, price of the product, a link to a URL, etc. Hence, the method disclosed in the present disclosure addresses the issue of counterfeit alternative leather products by embedding invisible identification markers within the authentic alternative leather material. Such identification markers, when detected, provide a verifiable link to the original product's identity, ensuring the authenticity of the product.

While this specification contains many specifics, these should not be construed as limitations on the scope of the disclosure or of what may be claimed, but rather as descriptions of features specific to particular implementations. Certain features that are described in this specification in the context of separate implementations may also be implemented in combination in a single implementation. Conversely, various features that are described in the context of a single implementation may also be implemented in multiple implementations separately or in any suitable sub-combination. Moreover, although features may be described above as acting in certain combinations and even initially claimed as such, one or more features from a claimed combination may in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products.

A number of implementations have been described. Nevertheless, it will be understood that various modifications may be made without departing from the spirit and scope of the disclosure. For example, various forms of the flows shown above may be used, with steps re-ordered, added, or removed. Accordingly, other implementations are within the scope of the following claims.

We claim:

1. A product comprising:
at least one portion being formed from mycelium;
a near infrared (NIR) fluorescent material integrated into the mycelium; and
a predetermined NIR pattern defined by the NIR fluorescent material in the mycelium, wherein the predetermined NIR pattern is an identification mark readable by a NIR camera.

2. The product of claim 1, wherein the predetermined NIR pattern is at a predetermined location on the at least one portion of the product.

3. The product of claim 1, further comprising additional pieces of material formed from either mycelium without the NIR fluorescent material or another type of material.

4. The product of claim 1, wherein the NIR fluorescent material is at least one of: indocyanine green, a NIR dye, and/or a NIR fluorescent protein.

5. The product of claim 1, wherein the NIR fluorescent material is integrated into the mycelium using at least one of: a surface NIR fluorescent dye, a spray pattern of a NIR fluorescent dye on a surface of the mycelium, a paint pattern of a NIR fluorescent dye on a surface of the mycelium, a 3D printing of mycelium with a NIR fluorescent dye, a stamp pattern of a NIR fluorescent dye on a surface of the mycelium, co-forming layers of mycelium with a genetically modified NIR fluorescent mycelium, and/or biologically growing a NIR fluorescent pattern.

6. The product of claim 5, further comprising: a predetermined modification of the mycelium for greater absorption of the NIR fluorescent material.

7. The product of claim 6, wherein the predetermined modification is a surface modification using a chemical.

8. The product of claim 1, wherein the mycelium includes a mixture of conventional mycelium and at least one genetically modified specie of mycelium that express NIR fluorescent proteins, and producing a biological NIR fluorescent pattern, which forms the identification mark.

9. A method for creating a tag on or in mycelium, the method comprising:
forming a near infrared (NIR) pattern on or in the mycelium;
forming a mycelium leather from the mycelium, wherein the mycelium leather, in dependence on the forming of the mycelium leather, contains the NIR pattern or a modified NIR pattern; and
associating the NIR pattern or the modified NIR pattern as an identification mark operable to be read by a NIR camera.

10. The method of claim 9, wherein the forming the mycelium leather comprises three-dimensionally printing the mycelium into an object.

11. The method of claim 9, wherein forming the mycelium leather comprises growing the mycelium in a mold to create an object.

12. The method of claim 9, wherein forming the mycelium leather comprises compressing a block of mycelium into a thin sheet of mycelium leather.

13. The method of claim 12, wherein the forming the NIR pattern comprises:
integrating a NIR fluorescent dye into a surface of the mycelium to define a first pattern in the surface of the block of mycelium;
drying the NIR fluorescent dye; and
compressing the block of mycelium with the NIR fluorescent dye into the thin sheet of mycelium leather, wherein the compressing distorts the first pattern into a second pattern, which forms the identification mark.

14. The method of claim 13, further comprising:
cutting the thin sheet of mycelium leather into segments for assembly into a product, including cutting a segment of mycelium leather into a size and shape such that subsequent assembly of the product positions the second pattern at a predetermined location on the product; and
assembling the segments in a manner that the second pattern appears in the predetermined location of the product.

15. The method of claim 13, further comprising:
cutting the thin sheet of mycelium leather into segments for assembly into a product, including cutting a segment of mycelium leather into a size and shape such that subsequent assembly of the product positions the second pattern at a predetermined orientation on the product; and
assembling the segments in a manner that the second pattern appears in the predetermined orientation of the product.

16. The method of claim 13, further comprising:
maintaining in a database the second pattern relative to the thin sheet of mycelium leather;
receiving a NIR image of a product;
cross referring the NIR image against the database; and
in response to the NIR image matching the second pattern, sending confirmation that the product is authentic.

17. The method of claim 13, wherein the NIR fluorescent dye is selected from a group including one or more of DIC proprietary NIR fluorescent dye, Terrylenimides, and/or Lumiprobe NIR fluorescent dyes.

18. The method of claim 9, wherein forming the NIR pattern comprises at least one of: a surface NIR fluorescent dye, a spray pattern of a NIR fluorescent dye on a surface of the mycelium, a paint pattern of a NIR fluorescent dye on a surface of the mycelium, a 3D printing of mycelium with a NIR fluorescent dye, a stamp pattern of a NIR fluorescent dye on a surface of the mycelium, co-forming layers of mycelium with a genetically modified NIR fluorescent mycelium, and/or biologically growing a NIR fluorescent pattern.

19. The method of claim 9, further comprising pretreating a surface of the mycelium with a chemical to improve the rate of absorption of an NIR fluorescent dye, wherein the chemical is one or more of crosslinked polyethylenimine, sulfuric acid, and/or sodium hydroxide.

20. A method for creating tag on or in mycelium, the method comprising:
forming a mycelium leather from mycelium;
forming a near infrared (NIR) pattern on or in the mycelium leather; and
associating the NIR pattern as an identification mark operable to be read by a NIR camera.
